(19) United States
(12) Patent Application Publication
(10) Pub. No.: US 2022/0301664 A1
(43) Pub. Date: Sep. 22, 2022

(54) AUTOMATED MONITORING OF CLINICAL DATABASE ELEMENTS

(71) Applicant: Evernorth Strategic Development, Inc., St. Louis, MO (US)

(72) Inventors: Myrna Belingon, Cary, NC (US); Arvind R. Sixtus, Fair Lawn, NJ (US); Ronald Buesser, Boonton Township, NJ (US); Jonathan Ehlen, Orlando, FL (US); Kenneth J. Catalanotto, St. Louis, MO (US); Heather-Ann Falcon, Mahwah, NJ (US); Marian Wilson, Fair Lawn, NJ (US); Mark D. Wong, Chesterfield, MO (US)

(21) Appl. No.: 17/202,507

(22) Filed: Mar. 16, 2021

(51) Int. Cl. G16H 10/60; G16H 40/20; G06F 9/54

(52) U.S. Cl. CPC G16H 10/60; G16H 40/20; G06F 9/547

(57) ABSTRACT

A computer system includes memory and processor hardware configured to execute instructions including obtaining structured patient data specific to a patient entity from a patient database, obtaining structured enterprise data specific to the patient entity from an enterprise database, obtaining structured enrollment data specific to the patient entity from an enrollment database, processing the obtained data to generate structured patient insights data associated with the patient entity, and storing the structured patient insights data in a patient insight data structure of an insights database for access by a user device via an API. The instructions include determining a subset of the multiple patient entities associated with a client entity, processing structured data associated with the subset of the multiple patient entities to generate structured client insight data, and storing the structured client insight data in a client insight data structure of the insights database for access via the API.

### FIELD

The present disclosure relates to computer-automated database operations, and more particularly to automated monitoring of clinical database elements.

### BACKGROUND

Healthcare providers, health insurance plan providers, monitoring device vendors, and organizations that offer employer-sponsored plans to their employees, may periodically look up health status information for various patients within their member groups. In some situations, patients may be contacted in an attempt to improve the patient's prescription drug adherence, or other health status. User interfaces may display patient health status information for view by an administrator or healthcare provider.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computer system includes memory hardware configured to store computer-executable instructions, and processor hardware configured to execute the instructions. The instructions include obtaining a set of multiple patient entities, and for each patient entity in the set of the multiple patient entities, obtaining structured patient data specific to the patient entity from a patient database configured to store patient data structures specific to multiple patient entities, obtaining structured enterprise data specific to the patient entity from an enterprise database configured to store enterprise data structures associated with the multiple patient entities, obtaining structured enrollment data specific to the patient entity from an enrollment database configured to store enrollment data structures associated with the multiple patient entities, processing the structured patient data, the structured enterprise data, and the structured enrollment data, to generate structured patient insights data associated with the patient entity, and storing the structured patient insights data in a patient insight data structure of an insights database for access by a user device via an application programming interface (API). The instructions include obtaining a set of multiple client entities, and for each client entity in the set of the multiple client entities, determining a subset of the multiple patient entities associated with the client entity, processing structured patient data, structured enterprise data, and structured enrollment data associated with the subset of the multiple patient entities to generate structured client insight data, and storing the structured client insight data in a client insight data structure of the insights database for access by the user device via the API.

In other features, the computer system includes the patient database, the enterprise database, the enrollment database, and the insights database. In other features, the obtaining structured patient data includes obtaining at least one of structured patient care data specific to the patient entity, obtaining structured remote monitoring data specific to the patient entity, and obtaining prescription drug claims data specific to the patient entity, the obtaining structured enterprise data includes at least one of obtaining structured demographic data specific to the patient entity, obtaining structured engagement data specific to the patient entity, and obtaining clinical intervention data specific to the patient entity, and the obtaining structured enrollment data includes obtaining structured performance guarantee data specific to at least of the multiple client entities associated with the patient entity.

In other features, the computer system includes a mid-tier aggregation layer in communication between the insights database and the user device. The mid-tier aggregation layer is configured to supply data to the user device from at least one database other than the insights database. In other features, the computer system includes a pass-through API gateway firewall located between the user device and the mid-tier aggregation layer.

In other features, the computer system includes the user device. The user device includes a user interface configured to receive a user input selection of a clinical insights view or a client performance guarantee view. The instructions include, in response to the selection being the clinical insights view, obtaining a subset of the patient insight data structures from the insights database, and displaying the subset of the patient insight data structures via the user interface, and in response to the selection being the client performance guarantee view, obtaining a subset of the client insight data structures from the insights database, and displaying the subset of the client insight data structures via the user interface.

In other features, the displaying the subset of the patient insight data structures includes displaying multiple patient sub-populations on the user interface. A displayed size of each of the multiple patient sub-populations corresponds to a relative size of a number of the multiple patient entities belonging to the patient sub-population. The instructions include receiving a user input selection of one of the multiple patient sub-populations, and displaying multiple groups of the selected patient sub-population on the user interface. A displayed size of each group corresponds to a relative size of a number of the multiple patient entities belonging to the group.

In other features, the displaying multiple groups includes receiving a user input selection of one of the multiple groups, displaying a patient entity list including each of the multiple patient entities belonging to the selected one of the multiple groups, receiving a user input selection of one of the multiple patient entities of the patient entity list, and displaying outreach data for the selected one of the multiple patient entities. In other features, the displaying the subset of the client insight data structures includes displaying multiple performance guarantee entries on the user interface. Each performance guarantee entry includes a displayed progress towards a performance guarantee target value specific to one of the multiple client entities. The instructions include receiving a user input selection of one of the multiple performance guarantee entries, and displaying multiple groups of the selected performance guarantee entry. A displayed size of each group corresponds to a relative size of a number of the multiple patient entities belonging to the group.

In other features, the multiple performance guarantee entries include at least one of a level of prescription drug adherence for patient entities belonging to one of the multiple client entities associated with one of performance guarantee entries, and a diabetes monitoring status for patient entities belonging to one of the multiple client entities associated with one of performance guarantee entries. In other features, the storing the structured patient insight data includes transferring the structured patient insight data from a Teradata database to a Postgres database via a Talend platform, on a scheduled periodic basis.

A computerized method for automated monitoring of clinical database elements includes obtaining a set of multiple patient entities, and for each patient entity in the set of the multiple patient entities, obtaining structured patient data specific to the patient entity from a patient database configured to store patient data structures specific to multiple patient entities, obtaining structured enterprise data specific to the patient entity from an enterprise database configured to store enterprise data structures associated with the multiple patient entities, obtaining structured enrollment data specific to the patient entity from an enrollment database configured to store enrollment data structures associated with the multiple patient entities, processing the structured patient data, the structured enterprise data, and the structured enrollment data, to generate structured patient insights data associated with the patient entity, and storing the structured patient insights data in a patient insight data structure of an insights database for access by a user device via an application programming interface (API). The method includes obtaining a set of multiple client entities, and for each client entity in the set of the multiple client entities, determining a subset of the multiple patient entities associated with the client entity, processing structured patient data, structured enterprise data, and structured enrollment data associated with the subset of the multiple patient entities to generate structured client insight data, and storing the structured client insight data in a client insight data structure of the insights database for access by the user device via the API.

In other features, the obtaining structured patient data includes obtaining at least one of structured patient care data specific to the patient entity, obtaining structured remote monitoring data specific to the patient entity, and obtaining prescription drug claims data specific to the patient entity, the obtaining structured enterprise data includes at least one of obtaining structured demographic data specific to the patient entity, obtaining structured engagement data specific to the patient entity, and obtaining clinical intervention data specific to the patient entity, and the obtaining structured enrollment data includes obtaining structured performance guarantee data specific to at least of the multiple client entities associated with the patient entity.

In other features, a mid-tier aggregation layer is in communication between the insights database and the user device. The method includes supplying, by the mid-tier aggregation layer, data to the user device from at least one database other than the insights database. In other features, the user device includes a user interface, and the method includes receiving, by the user interface, a user input selection of a clinical insights view or a client performance guarantee view, in response to the selection being the clinical insights view, obtaining, by the user interface, a subset of the patient insight data structures from the insights database, and displaying, by the user interface, the subset of the patient insight data structures via the user interface, and in response to the selection being the client performance guarantee view, obtaining, by the user interface, a subset of the client insight data structures from the insights database, and displaying, by the user interface, the subset of the client insight data structures via the user interface.

In other features, the displaying the subset of the patient insight data structures includes displaying multiple patient sub-populations on the user interface. A displayed size of each of the multiple patient sub-populations corresponds to a relative size of a number of the multiple patient entities belonging to the patient sub-population. The method includes receiving a user input selection of one of the multiple patient sub-populations, and displaying multiple groups of the selected patient sub-population on the user interface. A displayed size of each group corresponds to a relative size of a number of the multiple patient entities belonging to the group.

In other features, the displaying multiple groups includes receiving a user input selection of one of the multiple groups, displaying a patient entity list including each of the multiple patient entities belonging to the selected one of the multiple groups, receiving a user input selection of one of the multiple patient entities of the patient entity list, and displaying outreach data for the selected one of the multiple patient entities. In other features, the displaying the subset of the client insight data structures includes displaying multiple performance guarantee entries on the user interface. Each performance guarantee entry includes a displayed progress towards a performance guarantee target value specific to one of the multiple client entities. The method includes receiving a user input selection of one of the multiple performance guarantee entries, and displaying multiple groups of the selected performance guarantee entry. A displayed size of each group corresponds to a relative size of a number of the multiple patient entities belonging to the group.

In other features, the multiple performance guarantee entries include at least one of a level of prescription drug adherence for patient entities belonging to one of the multiple client entities associated with one of performance guarantee entries, and a diabetes monitoring status for patient entities belonging to one of the multiple client entities associated with one of performance guarantee entries. In other features, the storing the structured patient insight data includes transferring the structured patient insight data from a Teradata database to a Postgres database via a Talend platform, on a scheduled periodic basis.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example system for automated monitoring of clinical database elements.

FIG. 5 is a message sequence chart illustrating example interactions between components of the system of FIG. 4.

FIG. 6 is a flowchart depicting an example process for automated monitoring of clinical database elements.

FIG. 7A is a graphical illustration of an example user interface for displaying performance guarantee data elements.

FIG. 7B is a graphical illustration of an example sub-screen of the user interface of FIG. 7A.

FIG. 8A is a graphical illustration of an example user interface for displaying clinical data element groups.

FIG. 8B is a graphical illustration of an example sub-screen of the user interface of FIG. 8A.

FIG. 8C is a graphical illustration of an example element list for the user interface of FIG. 8A.

FIG. 8D is a graphical illustration of an example element detail view for the user interface of FIG. 8A.

FIG. 9 is a flowchart depicting an example process for navigating the example user interfaces illustrated in FIGS. 7A, 7B, 8A, 8B, 8C, and 8D.

In the drawings, reference numbers may be reused to identify similar and/or identical elements.

### DETAILED DESCRIPTION

### High-Volume Pharmacy

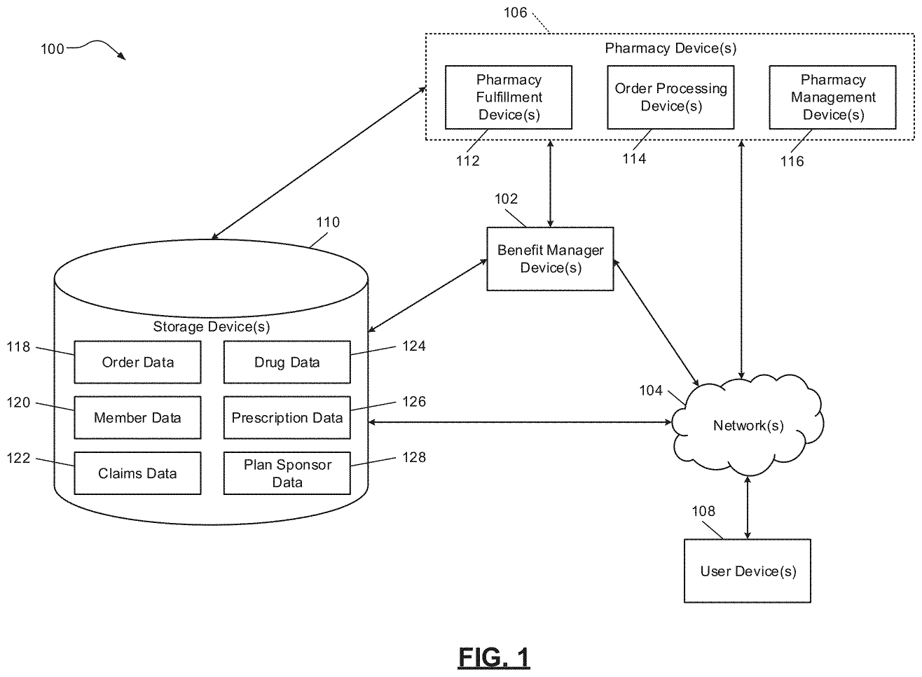

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Va.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

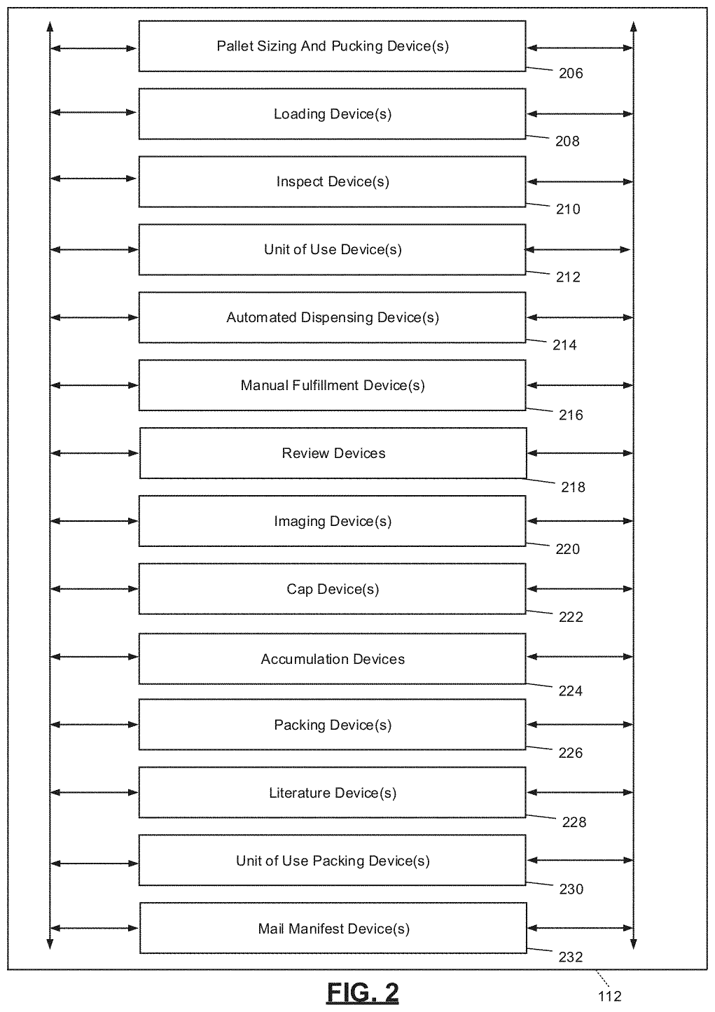

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

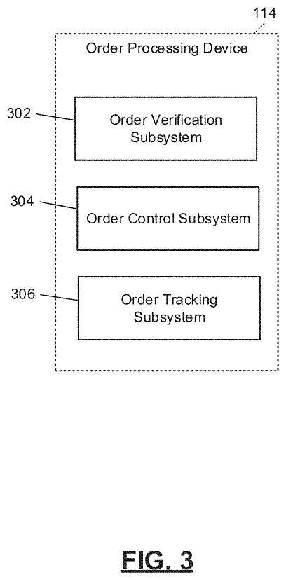

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### Automated Clinical Database Element Monitoring System

In various implementations, an automated clinical database element monitoring system allows clients (such as healthcare providers, health insurance plan providers, monitoring device vendors, and organizations that offer employer-sponsored plans to their employees) to use a care insights hub user interface to monitor the client's patient population (such as members enrolled in a health insurance plan offered by or sponsored by a client), look for clinical gaps and opportunities, and take action to perform patient outreaches.

The care insights hub user interface may include multiple dashboards for monitoring different aspects of the patient populations. For example, a performance guarantees dashboard may allow a client to monitor performance of members of the client's patient population, to compare prescription drug adherence or other metrics of the members to performance guarantees (such as target adherence levels or target health monitoring improvement value), in order to identify areas of concern. The user may select a clinical performance guarantee target, and view automatically generated health insights for sub-populations that are either meeting or not meeting the performance guarantee target.

In various implementations, a user may view clinical insights of patients independent of performance guarantee targets. For example, a user may select a clinical insights dashboard to uncover issues in specific patient sub-populations. The user may successively narrow down the sub-population by a number of demographic and clinical variables. The variables may be applied in any order, and the user can traverse forward and backward in their analysis, by adding or removing variables. Sub-populations may be presented on a screen of a user interface as multiple bubbles, with a size of each bubble representing the size of the patient sub-population that meets the selected criteria.

Once a user has narrowed down to a specific sub-population, the user may switch to a sub-population list to view the members in that sub-population. In various implementations, the user may select from up to thirty or more demographic and clinical fields to be displayed for each patient in the list. The user may further refine the sub-population list by applying a filter based on any of the displayed fields.

In various implementations, the user may drill down on a patient in the sub-population list, to proceed to an individual patient view that displays details of the patient, such as intervention opportunities, outreaches, referrals, medications, medical conditions, lab data, remote monitoring, referrals and other data. From the individual patient view, a user may perform a manual outreach, or refer a patient to another clinician. The user may traverse back to the sub-population list from the individual patient view, and back to the clinical insights bubble view from the sub-population list view.

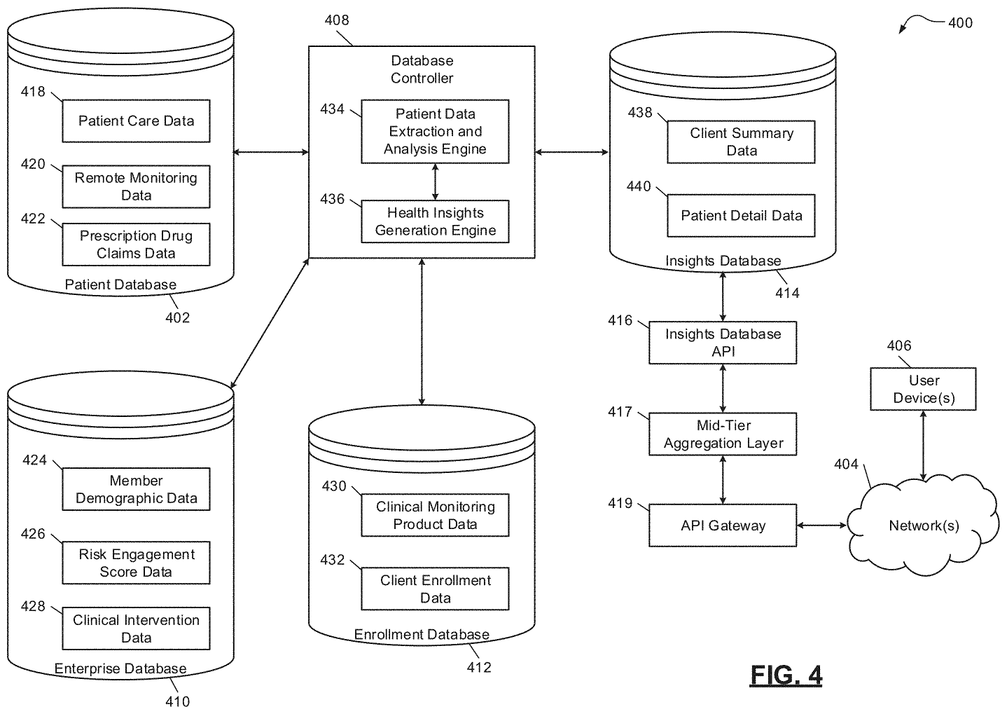

FIG. 4 is a functional block diagram of an example system 400 for automated monitoring of clinical database elements. The system 400 includes multiple databases, which may be deployed in a computer network system. The system 400 may include one or more servers, desktop computers, laptop computers, tablets, or smartphones.

As shown in FIG. 4, the system 400 includes a patient database 402 that stores patient care data 418, remote monitoring data 420, and prescription drug claims data 422. The patient care data 418, remote monitoring data 420, and prescription drug claims data 422 may be located in different physical memories within the patient database 402, such as different random access memory (RAM), read-only memory (ROM), a non-volatile hard disk or flash memory. In some implementations, the patient care data 418, remote monitoring data 420, and prescription drug claims data 422 may be located in the same memory (such as in different address ranges of the same memory).

In various implementations, the patient database 402 may include a Teradata database that obtains data from different sources. For example, the prescription drug claims data 422 may be obtained from a data store that includes information about proportion of days covered (PDC) claims associated with different patients. Remote monitoring data 420 may include data obtained from sensor devices that detect health parameters associated with the patient, such as glucose meters for diabetes patients. The patient care data 418 may include medical history records for various patients. Although FIG. 4 illustrates three types of data stored in the patient database 402, various implementations may include other suitable types of data associated with patient health information.

The system 400 includes an enterprise database 410 that stores member demographic data 424, risk engagement score data 426, and clinical intervention data 428. The member demographic data 424, risk engagement score data 426, and clinical intervention data 428 may be located in different physical memories within the enterprise database 410, or may be located in the same memory.

The enterprise database 410 may include a Teradata database that stores the enterprise data. For example, the member demographic data 424 may store any suitable demographic information (such as age and sex) for members of a group health plan. The risk engagement score data 426 may include data that is indicative of potential health risks for various members, such as a likelihood that the member will experience a negative health event within a specified future time period.

The clinical intervention data 428 may include data indicative of various intervention options to reduce the likelihood of the negative future health event for the member. For example, the clinical intervention data 428 may include data about various communication channels such as text, email, physical mail, etc., for contacting the user to address the potential health risk. Other example clinical intervention options may include automated phone calls, or live calls from a pharmacist or physician. The clinical intervention data 428 may include data about the success of the various types of interventions for one or more members according to historical interventions. Although FIG. 4 illustrates three types of data stored in the enterprise database 410, various implementations may include other suitable types of data (which may be associated with a health plan provider).

The system 400 also includes an enrollment database 412 that stores clinical monitoring data 430 and client enrollment data 432. The clinical monitoring data 430 and client enrollment data 432 may be located in different physical memories within the enrollment database 412, or may be located in the same memory.

In various implementations, clients (such as companies that offer employer-sponsored health insurance) may enroll to participate in various aspects of the automated monitoring performed by the system 400. For example, clients may select different health monitoring products offered for use in the system 400, such as monitoring adherence and non-adherence of prescription drug use by patients within the client's member group, or monitoring diabetes omission of care information. Clients may adjust configuration settings of their enrollment, such as start and end dates for monitoring, types of performance guarantees and levels that the client would like to have monitored, etc. For example, the system 400 may use a distributed streaming platform such as Apache Kafka to obtain enrollment and product offering data, which may be synchronized in a Hadoop database.

As shown in FIG. 4, the system 400 includes a database controller 408 that interfaces of the patient database 402, the enterprise database 410, and the enrollment database 412. The database controller 408 includes a patient data extraction and analysis engine 434. The patient data extraction and analysis engine 434 may obtain one or more of the patient care data 418, the remote monitoring data 420, and the prescription drug claims data 422 from the patient database 402, and process the obtained data according to one or more rules.

For example, the patient data extraction and analysis engine 434 may compile data about adherence of patients to prescription drug dosage plans, omission of care situations, care optimization status, etc. The compiled data may be stored in a Teradata staging database, which may be accessed by the health insights generation engine 436.

The health insights generation engine 436 may process data received from the patient database 402, the enterprise database 410, and the enrollment database 412. For example, the health insights generation engine 436 may receive data indicative of eligibility of patients for participation in one or more product offerings of the automated monitoring system 400, and process eligible patients according to the member demographic data 424 received from the enterprise database 410.

The health insights generation engine 436 may then determine one or more scores for each patient, such as a risk score, an engagement score, and a channel score, according to the risk engagement score data 426 received from the enterprise database 410. The health insights generation engine 436 may determine whether any historical intervention updates have occurred for each patient, according to the clinical intervention data 428 received from the enterprise database 410.

In various implementations, the health insights generation engine 436 may perform code mapping by mapping product identifiers with the clinical monitoring data 430 and the client enrollment data 432 obtained from the enrollment database 412. For example, the code mapping may be performed on the previously processed member demographics, risk score, engagement score, and score, channel score, and clinical intervention updates according to one or more rules specified for the health insights generation engine 436.

The health insights generation engine 436 interfaces within the insights database 414 that stores client summary data 438 and patient detail data 440. For example, the health insights generation engine 436 may supply processed data to staging tables in the insights database 414. A client summary staging table may create stored data that has been calculated or summarized by the health insights generation engine 436 at a group label level, while a patient detail staging table stores calculated or summarized data from the health insights generation engine 436 at an individual patient level.

In various implementations, each staging table supplies data to a Talend ETL insight platform, based on a nightly extraction process. A nightly refresh from the Talend ETL insight platform may update tables in the insights database 414. For example, the client summary data 438 may be stored in a Postgres table that provides a patient summary by client entity, while the patient detail data 440 is stored in a Postgres table that provides details by individual patient.

The client summary data 438 may include multiple parameters, such as an umbrella product description (for example, diabetes adherence), a status of whether specified criteria are met or not met, an age range subgroup, a gender subgroup, a status of whether remote monitoring is offered for a group, etc. The client summary data 438 may be summarized by a number of adherent patients within a client group, compared to a number of non-adherent patients in the client group. The patient detail data 440 may allow a user to drill down into various parameters related to individual patients, as described further below.

As shown in FIG. 4, the insights database 414 interfaces with the insights database API 416. For example, the insights database API 416 may provide access to the insights database 414, to allow data from the insights database 414 to be displayed on a user interface screen (such as a screen of the user device 106). In various implementations, the insights database API 416 may be used to validate whether a user is allowed to access data from the insights database 414, prior to sending information for display on a user interface screen.

FIG. 4 also illustrates the system 400 including a mid-tier aggregation layer 417 that interfaces with the insights database API 416. In various implementations, the mid-tier aggregation layer 417 may pull data from sources other than the insights database 414, such as user profiles, authentication credentials, references to external databases other than the insights database 414, etc. The mid-tier aggregation layer 417 may be configured to perform one or more functions on data transmitted to the user device 406, such as routing functions, calculation functions and formatting functions (for example, transforming data from disparate sources into a standardized format for a user interface of the user device 406).

The APA gateway 419 may be a pass through gateway, such as a firewall. The APA gateway 419 may allow the user device 406 to view insights from the insights database 414, via one or more networks 404. For example, an administrator may operate a user interface of the user device 406 to analyze clinical database elements. The user device 406 may include any suitable user device for displaying text and receiving input form a user, including a desktop computer, a laptop computer, a tablet, a smartphone, etc. The user device 406 may access the insights database 414 through one or more networks 404, the APA gateway 419, the mid-tier aggregation layer 417, and the insights database API 416. Example networks may include a wireless network, a local area network (LAN), the Internet, a cellular network, etc.

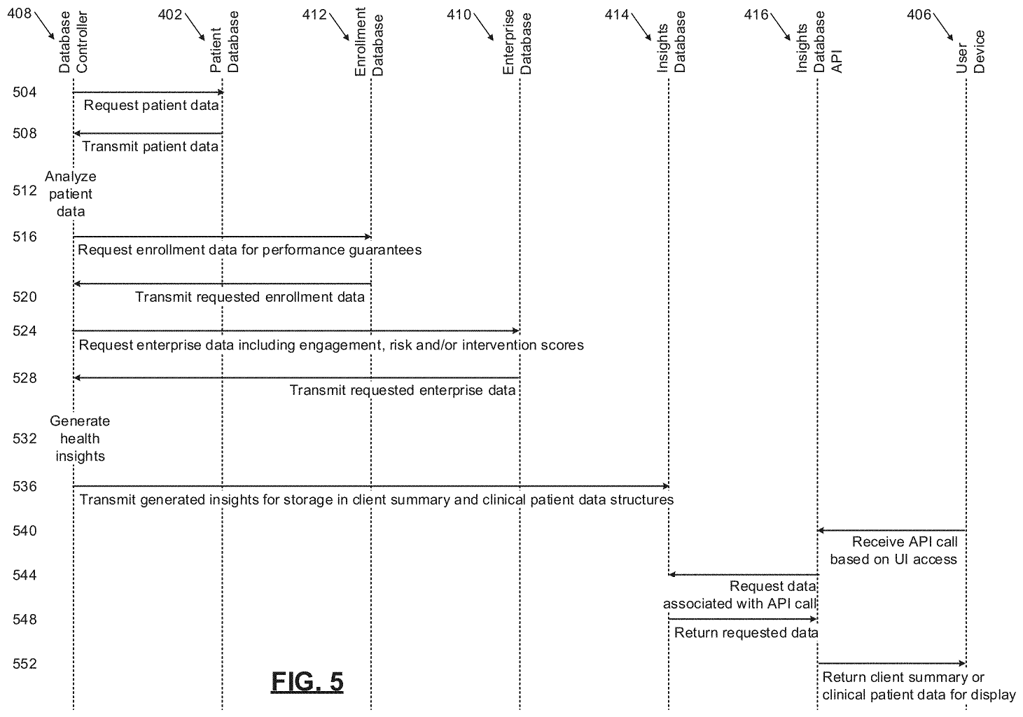

FIG. 5 is a message sequence chart illustrating example interactions between components of the system of FIG. 4. At line 504, the database controller 408 requests patient data from the patient database 402, such as the patient care data 418, the remote monitoring data 420, and the prescription drug claims data 422.

At line 508, the patient database 402 returns patient data to the database controller 408. The database controller 408 then analyzes the patient data at line 512. For example, the patient data extraction and analysis engine 434 may process the patient care data 418, the remote monitoring data 420, and the prescription drug claims data 422, according to one or more predefined rules or algorithms.

The database controller 408 requests enrollment data for performance guarantees from the enrollment database 412, at line 516. For example, the database controller 408 may obtain the clinical monitoring data 430 and the client enrollment data 432 from the enrollment database 412. In various implementations, each product may define one or more performance guarantees, and each performance guarantee may include one or more target values for members of a health insurance provider client that enrolls in the performance guarantee. For example, a performance guarantee may provide a target prescription drug adherence level for members of the client, a target improvement in diabetes health status of members of the client, etc. At line 520, the enrollment database 412 transmits the requested enrollment data to the database controller 408.

At line 524, the database controller requests enterprise data from the enterprise database 410, including engagement scores, risk scores, and/or interventions scores. For example, the database controller 408 may request the member demographic data 424, the risk engagement score data 426, and the clinical intervention data 428. At line 528, the enterprise database 410 transmits the requested enterprise data to the database controller 408.

The database controller 408 generates health insights at line 532. For example, the health insights generation engine 436 may process the data received from the patient database 402, the enrollment database 412, and the enterprise database 410, to generate the health insights data for multiple members of an enrolled client's group health plan. The generated health insights data may be determined according to any suitable rules, algorithms, etc. For example, the health insights generation engine 436 may determine a health status of various patients based on the data obtained from the patient database 402, identify products that a client including the patients is enrolled in according to the enrollment database 412, and determine risks for the patients and most likely successful engagement channels for the patients according to data obtained from the enterprise database 410.

At line 536, the database controller 408 transmits the generated insights to the insights database 414, for storage in client summary and clinical patient data structures. For example, the transmitted data may be stored in a data structure table of the client summary data 438, and a data structure table of the patient detail data 440. The data stored in data structure table(s) of the client summary data 438 may grouped to generate health insights data according to specified client groups, and the data structure table(s) of the patient detail data 440 may group data to generate health insights corresponding to individual patients.

The insights database API 416 receives an API call from the user device 406, at line 540, based on access to a user interface displayed on a screen of the user device 406. At line 544, the insights database API 416 requests data associated with the API call from the insights database 414. For example, the user may access the user interface on the user device 406 to view the status of a performance guarantee associated with a particular client, and the user device 406 may make an API call using insights database API 416 to obtain data related to the desired performance guarantee.

At line 548, the insights database 414 returns the requested data to the insights database API 416. The insights database API 416 then returns client summary data or clinical patient data for display on the user interface of the user device 406, at line 552. For example, the insights database 414 may supply portions of the client summary data 438 and/or the patient detail data 440 that corresponds to the user selection of elements, for view on the user interface of the user device 406.

### Automated Health Insights Generation

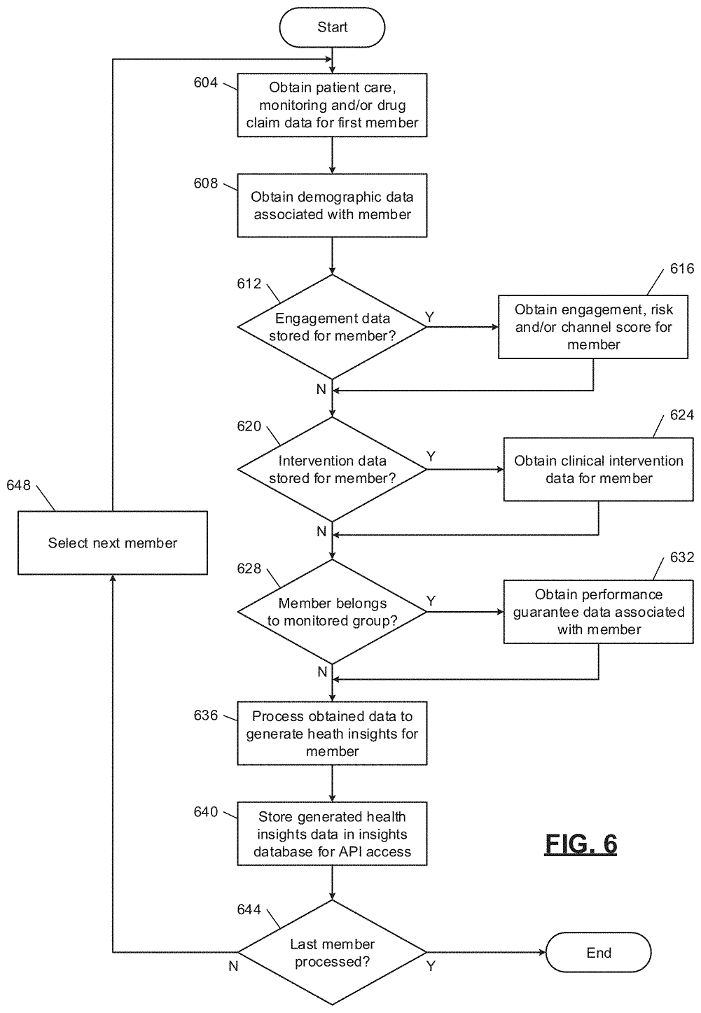

FIG. 6 is a flowchart depicting an example process for automated monitoring of clinical database elements. In various implementation, the example process of FIG. 6 may be implemented by the health insights generation engine 436 of the database controller 408.

At 604, control begins by obtaining patient care data, patient monitoring data, and/or patient drug claim data for a first member (such as a first member of a client group that provides health plans to members of the group). For example, control may obtain one or more of the patient care data 418, the remote monitoring data 420, and the prescription drug claims data 422, from the patient database 402.

Control proceeds to 608, to obtain demographic data associated with the member. For example, control may obtain member demographic data 424 specific to the first member, from the enterprise database 410. At 612, control determines whether engagement data is stored for the member. If so, control obtains engagement, risk, and/or channel score data for the member, at 616. For example, control may obtain this data from the enterprise database 410.

At 620, control determines whether invention intervention data is stored for the member. If so, control obtains intervention data for the member at 624. For example, control may obtain clinical intervention data 428 specific to the member, from the enterprise database 410. Control then proceeds to 628 to determine whether the member belongs to a monitored group. If so, control obtains performance guarantee data associated with the member at 632. For example, control may obtain clinical monitoring data 430 and client enrollment data 432 from the enrollment database 412, which is specific to the first member, or a group of the client that the first member belongs to.

Control then processes the obtained data to generate health insights for the member, at line 636. For example, control may determine whether the member is in compliance with one or more performance guarantees of a client group that the member belongs to. At 640, control stores the generated health insights data in an insights database for API access. For example, the generated health insights data may be stored in the insights database 414, so the user device 406 can obtain health insights data from the insights database 414 to be displayed on a user interface. At 644, control determines whether last member of a set of members has been processed. If not, control proceeds to 648 to select the next member, and returns to 604 to obtain patient care, monitoring, and/or drug claim data for selected next member.

### User Interface Navigation

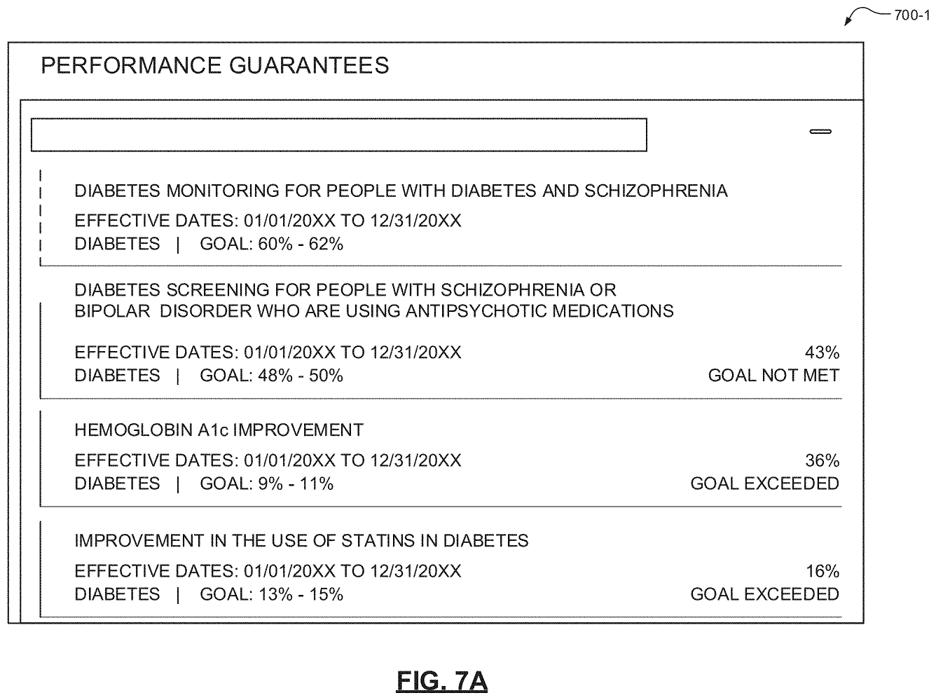

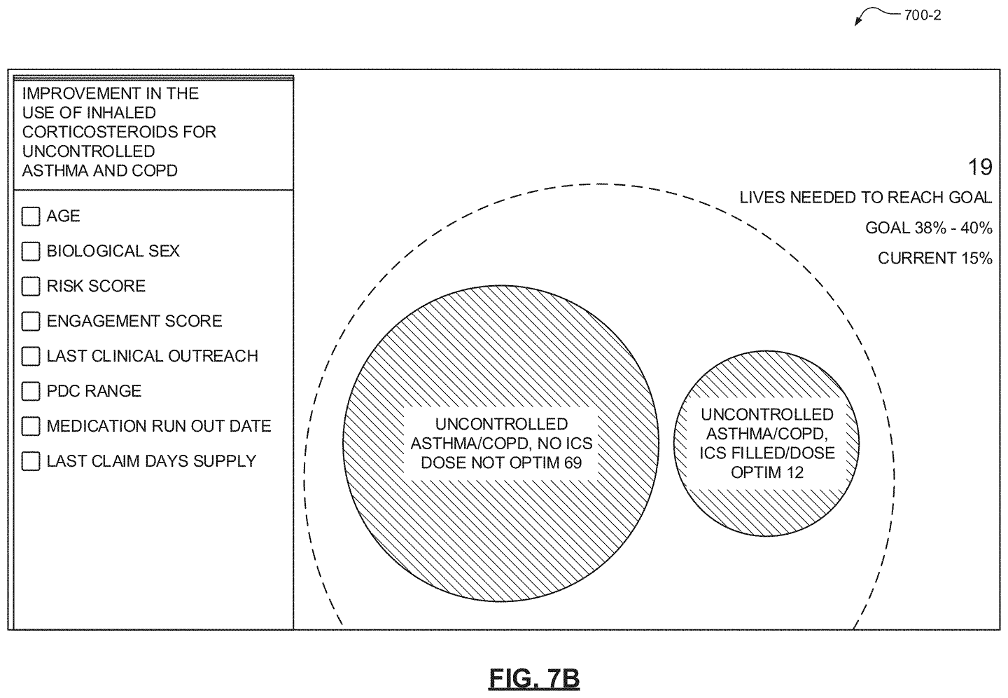

FIG. 7A is a graphical illustration of an example user interface 700-1 for displaying performance guarantee data elements. In various implementations, the user interface 700-1 may be displayed on the user device 406. As shown in FIG. 7A, multiple performance guarantees are displayed for selection by a user, to view additional details of patients included in the selected performance guarantee group.

For example, the first listed performance guarantee is diabetes monitoring for people with diabetes and schizophrenia. The entry indicates a specified effective data of the automated monitoring for this performance guarantee, which occurs from a specified start date to a specified end date. The displayed goal of 60% to 62% may indicate that the performance guarantee aims to achieve diabetes monitoring on a specified periodic basis for 60% to 62% of members of a group that a client has enrolled to the performance guarantee. For example, a performance guarantee product offering may include supplying glucose meters to members of the client group, where the performance guarantee provides a target value of 60% to 62% of all members using the glucose meter at least three times a week. In various implementations, other suitable monitoring frequencies may be used.

As another example, the second listed performance guarantee element is directed to diabetes screening for people with schizophrenia or bipolar disorder who are using antipsychotic medications. An example goal may include having 48% to 50% of members performing diabetes screening on a specified periodic basis. As shown on the right side of the second element, a current status may be only 43% of members performing sufficient diabetes screening, which means the goal is not currently met for this performance guarantee.

The third listed performance guarantee example is directed to hemoglobin A1c improvement. For example, a target improvement goal for members of the group may be a 9% to 11% improvement in A1c values for the group. In this example, a 36% improvement has currently occurred for members of a group, indicating that the performance guarantee target has been exceeded. Similarly, the bottom listed performance guarantee is directed to improvement in the use of statins in diabetes, and the 16% current value exceeds the target goal of 13% to 15% for the members of the group that is enrolled in the performance guarantee.

FIG. 7B illustrates an example user interface 700-2, which may be displayed if the user selects one of the performance guarantee elements from the user interface 700-1 of FIG. 7A. As shown in FIG. 7B, the user interface 700-2 is directed to improvement in use of inhaled corticosteroids for uncontrolled asthma and coping. The left-hand side of the screen includes various labels that may be used to filter the data that is displayed on the screen. For example, filter elements may include, but are not limited to, age, biological sex, risk score, engagement score, last clinical outreach, PDC range, medication run out date, and last claim supply.

The health insights data may be displayed on the screen of user interface 700-2 using circles (for example, bubbles) that have a size corresponding to a number of members within each group. For example, there are 69 members of a group defined as having uncontrolled Asthma/COPD, with no ICS and a non-optimal dose. There are 12 members of a group defined as having uncontrolled Asthma/COPD with ICS filled and an optimal dose. Therefore, the non-optimal group is shown with a larger circle than the optimal dose group.

In the upper right corner of the user interface 700-2, current progress towards the performance guarantee target is illustrated. For example, if the goal is 38% to 40% improvement, and a current number of members meeting the goal is only 15% of the group total, there may be a need for 19 additional members to meet specified improvement criteria in order to reach the target value of 38% to 40%.

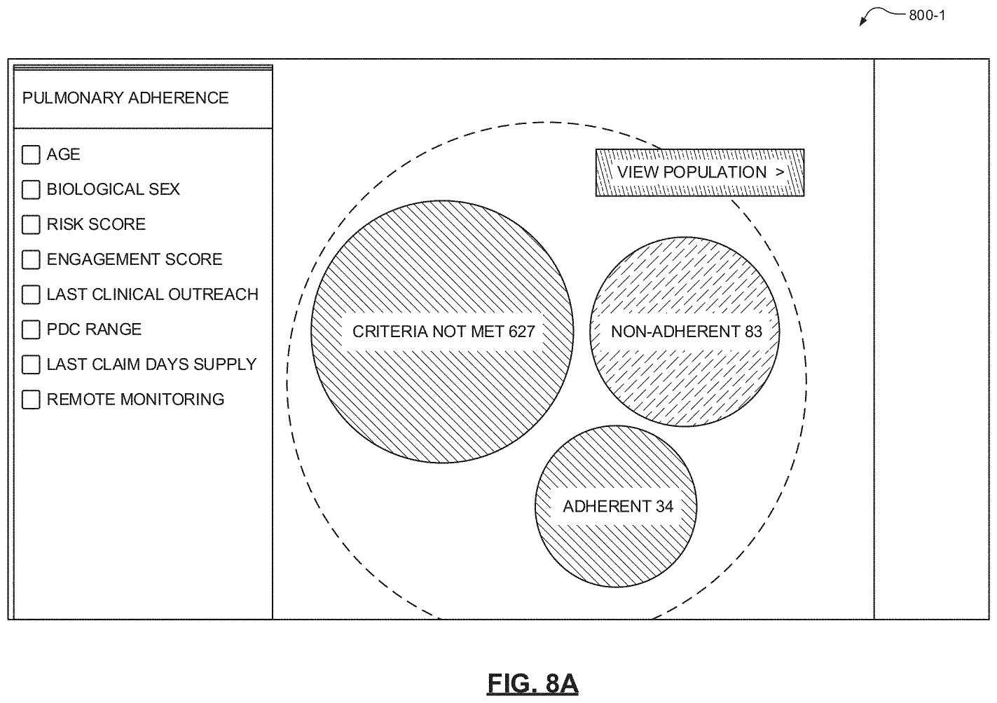

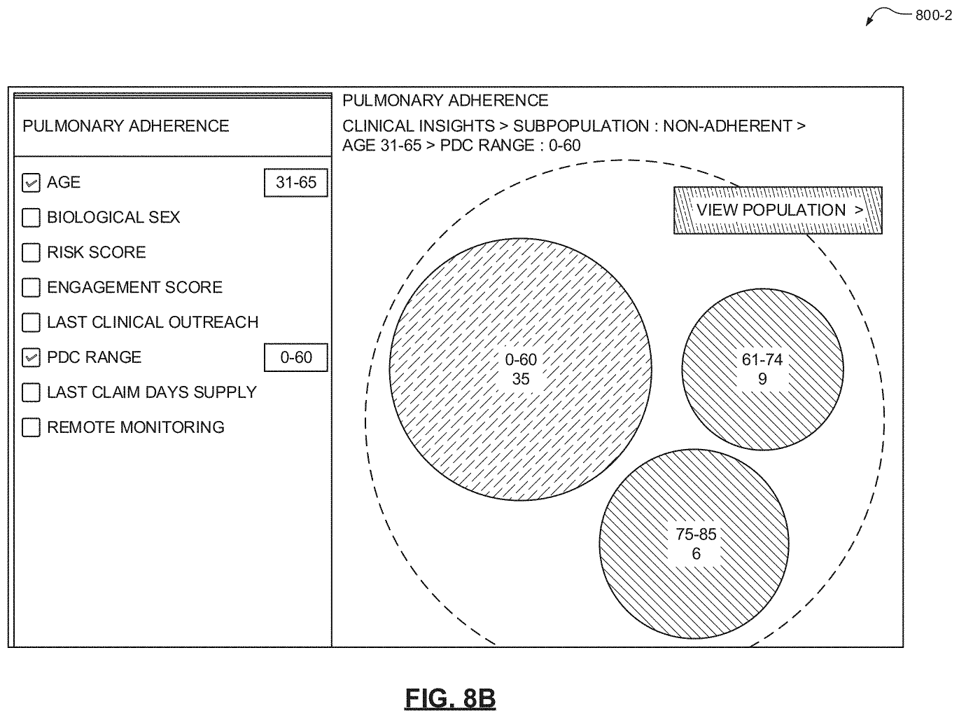

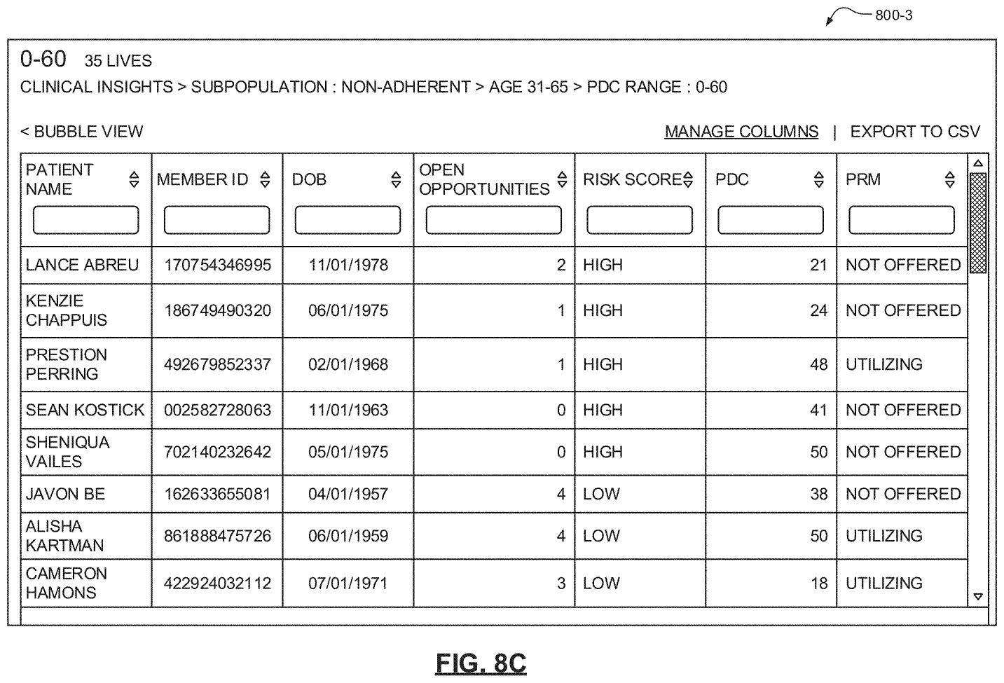

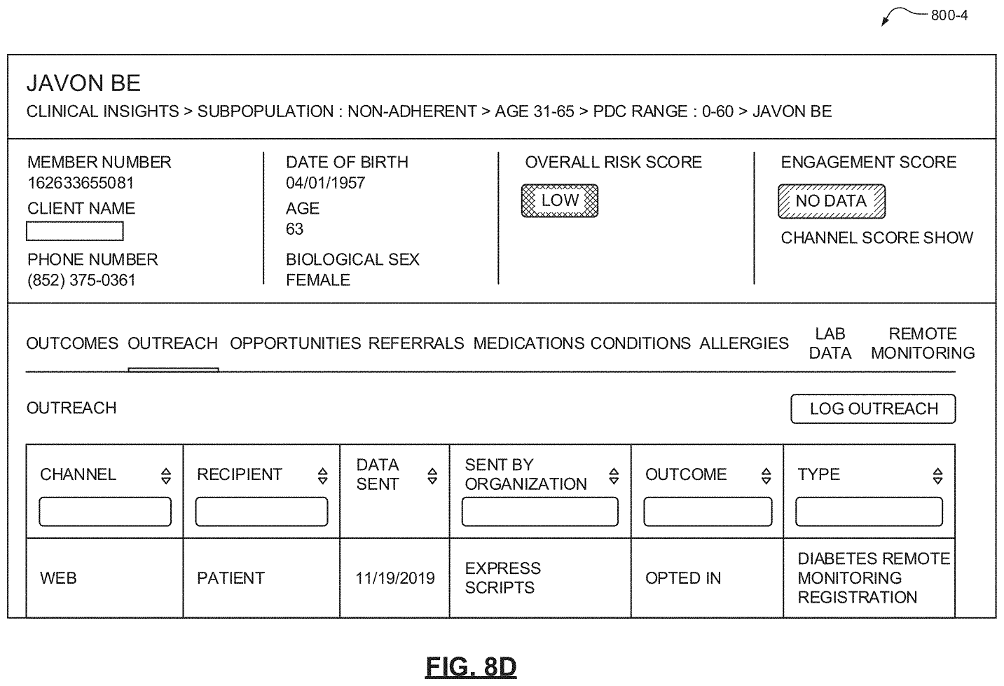

FIG. 8A is a graphical illustration of an example user interface 800-1 for displaying clinical data element groups. While the example user interface 700-1 of FIG. 7A illustrated example performance guarantees for enrolled client groups, the user interface 800-1 may be directed to displaying clinical data insights for a variety of individual patients.

For example, the user interface 800-1 is directed to pulmonary adherence, and includes various filter options on the left side of the screen. In this example, there are 627 patients for which pulmonary adherence criteria is not met, 83 members in the pulmonary non-adherence category, and 34 members in the pulmonary adherence category. As mentioned above, the sizes of the different groups may be larger or smaller relative to another, to assist the viewer in visually determining proportions or ratios of users that are adherent versus non-adherent. A user may click on one of the displayed elements to drill down and view further details of the selected element.

FIG. 8B illustrates an example user interface 800-2, which may be displayed if the user selects the non-adherent group from the user interface 800-1 of FIG. 8A. As shown in FIG. 8B, the non-adherent group is further filtered by an age range of 31 to 65 years old, and a PC range of 0 to 60.

In the example of FIG. 8B, the user interface 800-2 illustrates that within the age range of 31 to 65 years old, there are 35 members in the PDC range from 0 to 60. There are also nine members in the PDC range of 61 to 74, and six members in the PDC range 75 to 85. Again, the sizes of the displayed elements correspond to the relative numbers of members in each group.

FIG. 8C illustrates an example user interface 800-3, which may be displayed if a user selects one of the elements of the user interface 800-2 of FIG. 8B. For example, the user interface 800-3 corresponds to a user selection of the 0 to 60 PDC range element from the user interface 800-2 of FIG. 8B.

The user interface 800-3 includes an example element list of patients within the selected group. This provides a quick and easy way for an administrator, physician, nurse, etc. to quickly navigate to desired populations of members, based on generated health insights data about the members. As shown in FIG. 8C, each entry in the data structure table includes a patient name, a member ID, and a date of birth.

Each entry includes an open opportunities column, which may specify a number of opportunities for reaching out to the patient via one or more intervention channels. The structured patient detail list also includes a risk score column, a PDC column, and a PRM column. The risk score may be indicative of the likelihood that the member will experience an adverse health event with a future specified time period, such as one year.

FIG. 8D illustrates a further drill down in the user interface 800-4, which may be displayed if the user selects one of the patient entries from the element list database of the user interface 800-3 of FIG. 8C. For example, the patient details illustrated in the user interface 800-4 include a member number of the selected patient, and a client name of a group that provides a health insurance plan to the patient.

The entry of the user interface 800-4 also includes other patient data such as a phone number, a date of birth, an age, and a biological sex. An overall risk score for the patient may be displayed, based on generated health insights from the insights database 414. Similarly, an engagement score and channel score may be displayed to show one or more likelihoods that various intervention channels may change the behavior of the member, to successfully facilitate the member becoming adherent for one or more performance guarantee targets.

The user interface 800-4 also includes different tabs for outcomes, outreach, opportunities, referrals, medications, conditions, and allergies. An outreach portion at the bottom of the user interface 800-4 may be used to filtering historical outreaches to the patient, including an indication of the type of channel used, the recipient of the outreach data, a date that the outreach data was sent, the organization that sent the outreach data, the outcome of the outreach, and a type of the outreach outcome.

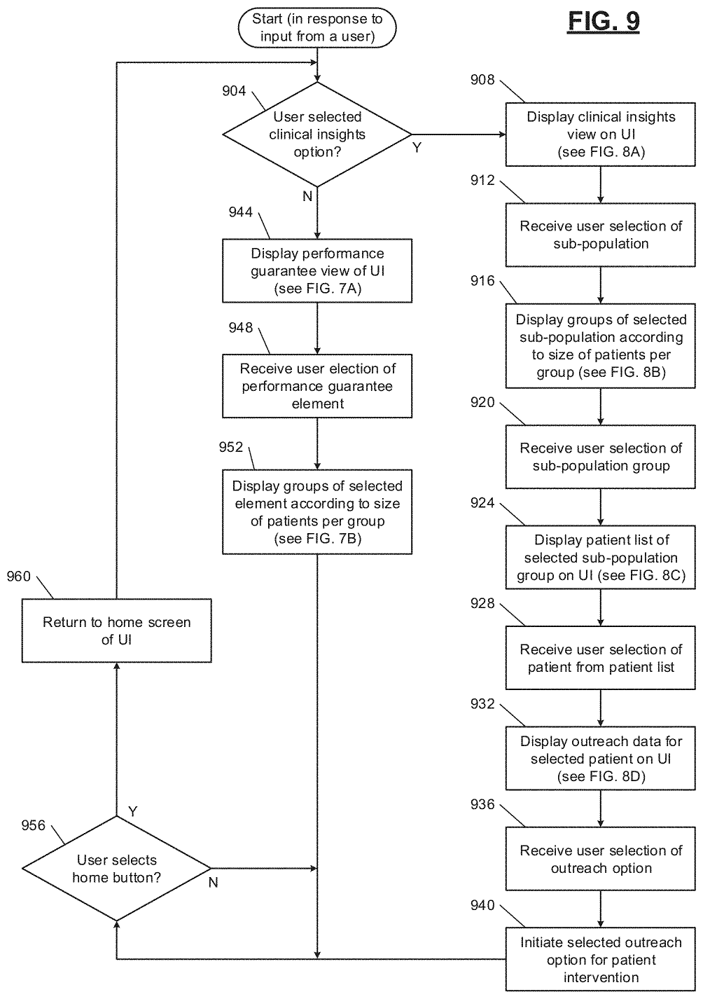

FIG. 9 is a flowchart depicting an example process for navigating user interfaces, such as the example user interfaces illustrated in FIGS. 7A, 7B, 8A, 8B, 8C and 8D. The process of FIG. 9 may be started in response to input from a user on the user interface of a screen of the user device 406. At 904, control determines whether the user selected a clinical insights option. For example, the user may be allowed to decide if they would prefer to look at clinical insights for individual patients, or if they would prefer to look at one or more performance guarantees monitored by the system.

If control determines at 904 that the user selected the clinical insights 9option, control proceeds to 908 to display a clinical insights view on the user interface. For example, an example clinical insights view is displayed in FIG. 8A. At 912, control receives a user selection of a sub-population displayed on the user interface. At 916, control displays groups of the selected sub-population, with a size of each group varied according to a relative number of members of each group. An example of displaying groups of the selected sub-population is illustrated in FIG. 8B, as described above.

At 920, control receives a user selection of one of the displayed sub-population groups. At 924, control displays a patient list of the selected sub-population group on the user interface. An example patient list is illustrated in FIG. 8C, as described above. Control then receives a user selection of one of the patients from the list at 928, and displays outreach data for the selected patient on the user interface at 932. An example patient detail screen is illustrated in FIG. 8D, as described above. At 936, control receives a user selection of an outreach option, then initiates the selected outreach option for the patient intervention at 940 (for example, by generating an email or text, or scheduling an automated or live call to the patient).

Control then determines whether the user has selected a home button, at 956. If not, control role continues to wait until the user selects navigation to a different screen. Once the user selects the home button at 956, control proceeds to return to the home screen of user face (or move back one level in the interface screens), at 960.

If the user does not select a clinical insights option at 904 (for example, because the user wants to view performance guarantee data), control proceeds to 944 to display a performance guarantee view of the user interface. An example performance guarantee view is illustrated in FIG. 7A, as described further above.

At 948, control receives a user selection of one of the displayed performance guarantee elements. At 952, control displays groups of the selected performance guarantee element, with a size of each displayed group varying according to a relative number of members in each group. An example of a user interface displaying groups of a selected performance guarantee element is illustrated in FIG. 7B, as described further above.

At 956, control determines whether a user has selected a home button (or a back button). If not, control continues to wait until a user supplies another screen navigation option as an input. At 956 control determines whether user has selected the home button. Once the user selects the home button at 956, control proceeds to return to the home screen of user face, at 960.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase at least one of A, B, and C should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WWI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system comprising:
memory hardware configured to store computer-executable instructions; and
processor hardware configured to execute the instructions,
wherein the instructions include:
obtaining a set of multiple patient entities;
for each patient entity in the set of the multiple patient entities:
obtaining structured patient data specific to the patient entity from a patient database configured to store patient data structures specific to multiple patient entities;
obtaining structured enterprise data specific to the patient entity from an enterprise database configured to store enterprise data structures associated with the multiple patient entities;
obtaining structured enrollment data specific to the patient entity from an enrollment database configured to store enrollment data structures associated with the multiple patient entities;
processing the structured patient data, the structured enterprise data, and the structured enrollment data, to generate structured patient insights data associated with the patient entity;
storing the structured patient insights data in a patient insight data structure of an insights database for access by a user device via an application programming interface (API);
obtaining a set of multiple client entities; and
for each client entity in the set of the multiple client entities:
determining a subset of the multiple patient entities associated with the client entity;
processing structured patient data, structured enterprise data, and structured enrollment data associated with the subset of the multiple patient entities to generate structured client insight data; and
storing the structured client insight data in a client insight data structure of the insights database for access by the user device via the API.

2. The computer system of claim 1 further comprising the patient database, the enterprise database, the enrollment database, and the insights database.

3. The computer system of claim 1 wherein:
the obtaining structured patient data includes obtaining at least one of structured patient care data specific to the patient entity, obtaining structured remote monitoring data specific to the patient entity, and obtaining prescription drug claims data specific to the patient entity;
the obtaining structured enterprise data includes at least one of obtaining structured demographic data specific to the patient entity, obtaining structured engagement data specific to the patient entity, and obtaining clinical intervention data specific to the patient entity; and
the obtaining structured enrollment data includes obtaining structured performance guarantee data specific to at least of the multiple client entities associated with the patient entity.

4. The computer system of claim 1 further comprising a mid-tier aggregation layer in communication between the insights database and the user device, wherein the mid-tier aggregation layer is configured to supply data to the user device from at least one database other than the insights database.

5. The computer system of claim 4 further comprising a pass-through API gateway firewall located between the user device and the mid-tier aggregation layer.

6. The computer system of claim 1 further comprising the user device, wherein the user device includes a user interface configured to receive a user input selection of a clinical insights view or a client performance guarantee view, and the instructions include:
in response to the selection being the clinical insights view:
obtaining a subset of the patient insight data structures from the insights database; and
displaying the subset of the patient insight data structures via the user interface; and
in response to the selection being the client performance guarantee view:
obtaining a subset of the client insight data structures from the insights database; and
displaying the subset of the client insight data structures via the user interface.

7. The computer system of claim 6 wherein the displaying the subset of the patient insight data structures includes:
displaying multiple patient sub-populations on the user interface, wherein a displayed size of each of the multiple patient sub-populations corresponds to a relative size of a number of the multiple patient entities belonging to the patient sub-population;
receiving a user input selection of one of the multiple patient sub-populations; and
displaying multiple groups of the selected patient sub-population on the user interface, wherein a displayed size of each group corresponds to a relative size of a number of the multiple patient entities belonging to the group.

8. The computer system of claim 7 wherein the displaying multiple groups includes:
receiving a user input selection of one of the multiple groups;
displaying a patient entity list including each of the multiple patient entities belonging to the selected one of the multiple groups;
receiving a user input selection of one of the multiple patient entities of the patient entity list; and
displaying outreach data for the selected one of the multiple patient entities.

9. The computer system of claim 6 wherein the displaying the subset of the client insight data structures includes:
displaying multiple performance guarantee entries on the user interface, wherein each performance guarantee entry includes a displayed progress towards a performance guarantee target value specific to one of the multiple client entities;
receiving a user input selection of one of the multiple performance guarantee entries; and
displaying multiple groups of the selected performance guarantee entry, wherein a displayed size of each group corresponds to a relative size of a number of the multiple patient entities belonging to the group.

10. The computer system of claim 9 wherein the multiple performance guarantee entries include at least one of:
a level of prescription drug adherence for patient entities belonging to one of the multiple client entities associated with one of performance guarantee entries; and
a diabetes monitoring status for patient entities belonging to one of the multiple client entities associated with one of performance guarantee entries.

11. The computer system of claim 1 wherein the storing the structured patient insight data includes transferring the structured patient insight data from a Teradata database to a Postgres database via a Talend platform, on a scheduled periodic basis.

12. A computerized method for automated monitoring of clinical database elements, the method comprising:
obtaining a set of multiple patient entities;
for each patient entity in the set of the multiple patient entities:
obtaining structured patient data specific to the patient entity from a patient database configured to store patient data structures specific to multiple patient entities;
obtaining structured enterprise data specific to the patient entity from an enterprise database configured to store enterprise data structures associated with the multiple patient entities;
obtaining structured enrollment data specific to the patient entity from an enrollment database configured to store enrollment data structures associated with the multiple patient entities;
processing the structured patient data, the structured enterprise data, and the structured enrollment data, to generate structured patient insights data associated with the patient entity;
storing the structured patient insights data in a patient insight data structure of an insights database for access by a user device via an application programming interface (API);
obtaining a set of multiple client entities; and
for each client entity in the set of the multiple client entities:
determining a subset of the multiple patient entities associated with the client entity;
processing structured patient data, structured enterprise data, and structured enrollment data associated with the subset of the multiple patient entities to generate structured client insight data; and
storing the structured client insight data in a client insight data structure of the insights database for access by the user device via the API.

13. The method of claim 12 wherein:
the obtaining structured patient data includes obtaining at least one of structured patient care data specific to the patient entity, obtaining structured remote monitoring data specific to the patient entity, and obtaining prescription drug claims data specific to the patient entity;
the obtaining structured enterprise data includes at least one of obtaining structured demographic data specific to the patient entity, obtaining structured engagement data specific to the patient entity, and obtaining clinical intervention data specific to the patient entity; and
the obtaining structured enrollment data includes obtaining structured performance guarantee data specific to at least of the multiple client entities associated with the patient entity.

14. The method of claim 12 wherein:
a mid-tier aggregation layer is in communication between the insights database and the user device; and
the method further comprises supplying, by the mid-tier aggregation layer, data to the user device from at least one database other than the insights database.

15. The method of claim 12 wherein the user device includes a user interface, and the method further comprises:
receiving, by the user interface, a user input selection of a clinical insights view or a client performance guarantee view;
in response to the selection being the clinical insights view:
obtaining, by the user interface, a subset of the patient insight data structures from the insights database; and
displaying, by the user interface, the subset of the patient insight data structures via the user interface; and
in response to the selection being the client performance guarantee view:
obtaining, by the user interface, a subset of the client insight data structures from the insights database; and
displaying, by the user interface, the subset of the client insight data structures via the user interface.

16. The method of claim 15 wherein the displaying the subset of the patient insight data structures includes:
displaying multiple patient sub-populations on the user interface, wherein a displayed size of each of the multiple patient sub-populations corresponds to a relative size of a number of the multiple patient entities belonging to the patient sub-population;
receiving a user input selection of one of the multiple patient sub-populations; and
displaying multiple groups of the selected patient sub-population on the user interface, wherein a displayed size of each group corresponds to a relative size of a number of the multiple patient entities belonging to the group.

17. The method of claim 16 wherein the displaying multiple groups includes:
receiving a user input selection of one of the multiple groups;
displaying a patient entity list including each of the multiple patient entities belonging to the selected one of the multiple groups;
receiving a user input selection of one of the multiple patient entities of the patient entity list; and
displaying outreach data for the selected one of the multiple patient entities.

18. The method of claim 15 wherein the displaying the subset of the client insight data structures includes:
displaying multiple performance guarantee entries on the user interface, wherein each performance guarantee entry includes a displayed progress towards a performance guarantee target value specific to one of the multiple client entities;
receiving a user input selection of one of the multiple performance guarantee entries; and
displaying multiple groups of the selected performance guarantee entry, wherein a displayed size of each group corresponds to a relative size of a number of the multiple patient entities belonging to the group.

19. The method of claim 18 wherein the multiple performance guarantee entries include at least one of:
a level of prescription drug adherence for patient entities belonging to one of the multiple client entities associated with one of performance guarantee entries; and
a diabetes monitoring status for patient entities belonging to one of the multiple client entities associated with one of performance guarantee entries.

20. The method of claim 12 wherein the storing the structured patient insight data includes transferring the structured patient insight data from a Teradata database to a Postgres database via a Talend platform, on a scheduled periodic basis.
